(19) United States
(12) Patent Application Publication
(10) Pub. No.: US 2003/0060810 A1
(43) Pub. Date: Mar. 27, 2003

(54) Method and apparatus for treating and/or removing an undesired presence on the skin of an individual

(76) Inventors: Diego Syrowicz, New York, NY (US); Somak Chattopadhyay, New York, NY (US); Ramon Rodriguez, Chicago, IL (US); Guillermo Oropeza, Huixquilucan (MX); Jonathan Kisner, New York, NY (US); Hernan Mercado-Corujo, Westland, MI (US)

Correspondence Address: OSTROLENK FABER GERB & SOFFEN, 1180 AVENUE OF THE AMERICAS, NEW YORK, NY, 100368403

(21) Appl. No.: 09/784,726

(22) Filed: Feb. 15, 2001

Related U.S. Application Data
(60) Provisional application No. 60/182,952, filed on Feb. 16, 2000.

(51) Int. Cl. A61B 18/20; A61B 18/14

(52) U.S. Cl. 606/9; 606/36; 606/43

(57) ABSTRACT

An apparatus and method for treating an undesired presence on an individual includes a CPU coupled to a laser control unit and a robotic arm control unit. A scanning area or volume is defined on the individual that includes the undesired presence. The CPU controls the robotic arm to move the laser and camera about the scanning area or volume while the camera scans individual portions of the scanning area or volume. The CPU receives this information from the camera and determines, based on the scanning, whether a particular portion includes part of the undesired presence. When the particular portion includes part of the undesired presence, the CPU actuates the laser. This process continues for all portions of the scanning area or volume. In an alternative embodiment, the CPU controls the robotic arm control unit to scan the entire area or volume and determines locations where there is an undesired presence. The CPU then again moves through the area or volume and actuates the laser at locations which will treat these undesired presences.

### CROSS-REFERENCE RELATED APPLICATIONS

This application claims priority to provisional application No. 60/182,952 filed Feb. 16, 2000, the entirety of which is hereby incorporated by reference.

### BACKGROUND OF THE INVENTION

The invention relates to an undesired presence treatment apparatus and, more particularly, to an apparatus which detects the location of an undesired presence on an individual and automatically moves and controls a treatment device to treat the undesired presence.

Individuals frequently have cosmetically undesired presences on their skin which they wish to have treated or even removed (hereinafter collectively referred to as “treated”). Examples of such presences include hair, moles, tatoos, pimples, scar tissue, warts, freckles, or other skin aberrations.

One prior art technique for treating an undesired presence shown in U.S. Pat. Nos. 5,653,706 and 5,860,967 provides a visualization means disposed proximate to a synchronized laser. The visualization means is connected to a display so that an apparatus technician can ascertain where on an individual's skin a laser would be applied if actuated. The laser is then actuated and burns the hair and the hair root. Another prior art technique disclosed in FR 2 590 791A1 uses a computer to detect the presence of an abnormality on the skin of the user. Once the abnormality is detected, an apparatus technician guides a laser to the appropriate position to treat the abnormality.

In all of these prior art techniques, an apparatus technician is required to actually guide the laser device to a position where the laser can treat the undesired presence. Such techniques are imprecise, lengthy, inefficient and possibly damaging because they are dependent upon the placement of the laser by an individual—the apparatus technician. Since the laser is moved by a human apparatus technician, the placement of the laser is necessarily imprecise due to human error and so the energy output from the laser must be weakened to avoid damaging desired skin. This results in less than optimal radiation being applied to the undesired presence. Such suboptimal radiation means that additional treatments must be performed to yield satisfactory results. These techniques are also costly because they require the services of a highly trained apparatus technician to operate the respective devices.

Therefore, there exists a need in the art for a treatment apparatus and method which is more precise, efficient, and less costly than that available in the prior art.

### SUMMARY OF THE INVENTION

An apparatus and method for treating an undesired presence on an individual includes a CPU coupled to a laser control unit and a robotic arm control unit. A scanning area or volume is defined on the individual which includes the undesired presence. The CPU controls the robotic arm to move the laser and camera about the scanning area or volume while the camera scans individual portions of the scanning area or volume. The CPU receives this information from the camera and determines, based on the scanning, whether a particular portion includes part of the undesired presence. When the particular portion includes part of the undesired presence, the CPU actuates the laser. This process continues for all portions of the scanning area or volume. In an alternative embodiment, the CPU controls the robotic arm control unit to scan the entire area or volume and determines locations where there is an undesired presence. The CPU then again moves through the area or volume and actuates the laser at locations which will treat these undesired presences.

These aspects, as well as others, will become apparent upon reading the following disclosure and corresponding drawings.

### BRIEF DESCRIPTION OF THE DRAWINGS

For the purpose of illustrating the invention, there is shown in the drawings a form which is presently preferred, it being understood, however, that the invention is not limited to the precise arrangements and instrumentalities shown.

FIG. 1 is a diagram of an undesired presence treatment system in accordance with the invention;

FIG. 2 is block diagram illustrating the operation of a CPU 12 used in the system of FIG. 1; and

FIG. 3 is block diagram illustrating another embodiment of the operation of the CPU 12 used in the system of FIG. 1.

FIG. 4 is a diagram of another embodiment of an undesired presence treatment unit in accordance with the invention.

### DETAILED DESCRIPTION OF THE PREFERRED EMBODIMENTS

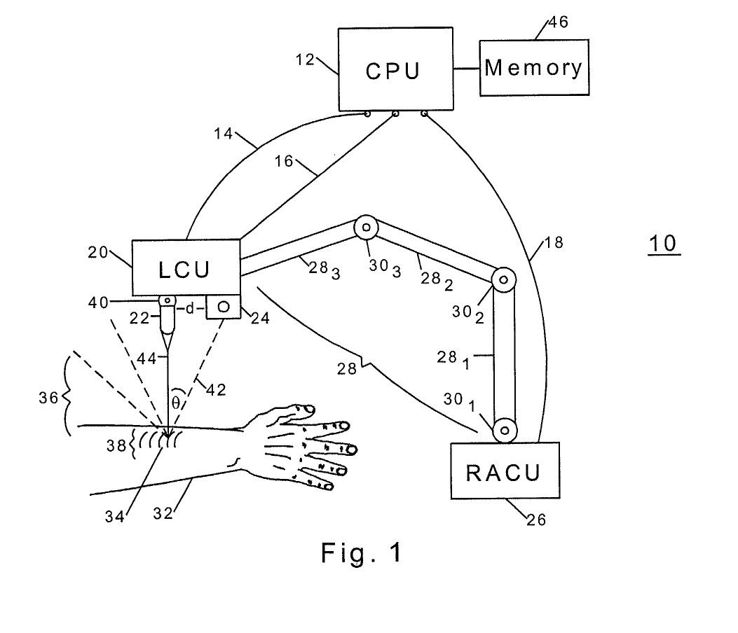

Referring to FIG. 1, there is shown an undesired presence treatment system 10 in accordance with the invention. A central processing unit (“CPU”) 12 controls the operation of system 10. CPU 12 is coupled to a memory 46. CPU 12 is also coupled to a laser and camera unit (“LCU”) 20 through cables 14 and 16 or by a wireless communications device. An undesired presence treatment unit or laser 22 and an image processor or camera 24 are disposed on laser and camera unit 20. Laser 22 can be, for example, an alexandrite laser. Although a laser is discussed, clearly other treatment devices may be used as is discussed more completely below. CPU 12 is also coupled to a robotic arm control unit 26 (“RACU”) through a cable 18 or wireless communications device. Robotic arm control unit 26 is mechanically and electrically coupled to a robotic arm 28 comprised of a plurality of arm sections, e.g., three robotic arm sections 28₁, 28₂, and 28₃ separated by joints 30₁, 30₂, and 30₃. Robotic arm section 28₃ is fixedly coupled to laser and camera unit 20. A laser joint 40 may be coupled between laser and camera unit 20 and laser 22 so that laser and camera unit 20 can control the movement of laser 22 with respect to laser and camera unit 20.

CPU 12 contains data for controlling robotic arm control unit 26 to move robotic arm 28, and thus laser and camera unit 20, to a treatment position with respect to a user's body part 32. In such a treatment position, laser 22 can be actuated to thereby treat an undesired presence 34 disposed upon or in body part 32. In the embodiment shown, hair is the undesired presence 34 though it should be clear that other undesired presences or abnormalities (e.g. moles, tatoos, pimples, scar tissue, warts, freckles, etc.) could also be treated with the invention.

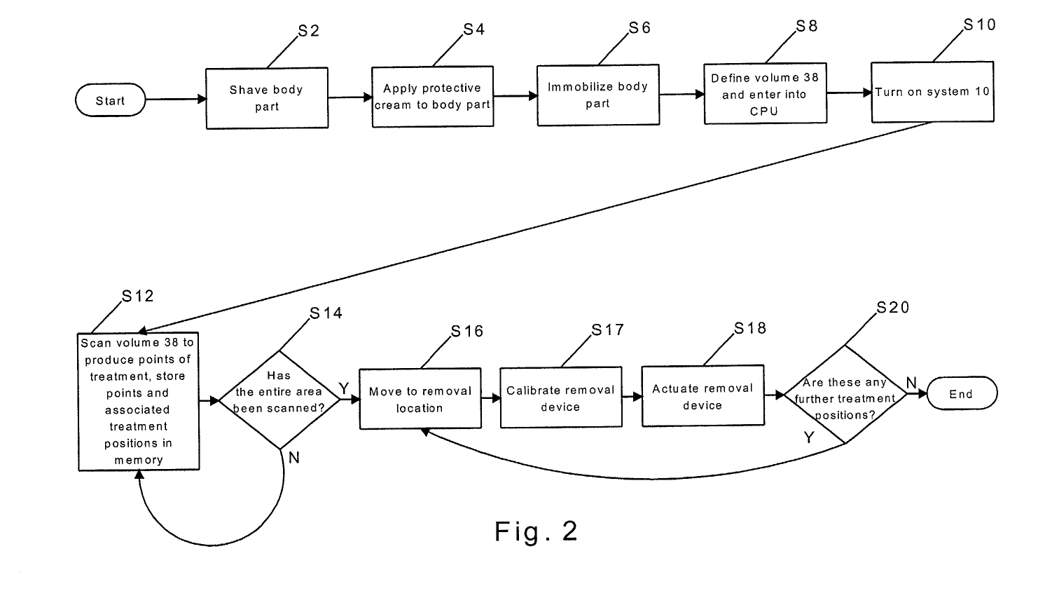

Referring now also to FIG. 2, the operation of system 10 will be explained. In use, at step S2, body part 32 is optionally shaved using conventional shaving techniques. At step S4, a protective cream or lotion may optionally be applied to the skin of body part 34. If the undesired presence is hair, any remaining hair extending beyond the individual's skin may be dyed (not shown in the figure) to increase the contrast between the hair and the surrounding skin. At step S6, a system technician immobilizes body part 32. At step S8, the system technician then defines a volume 38 within a coordinate system 36 where undesired presence 34 is located. The location of volume 38 within coordinate system 36 is entered into CPU 12. As the processing in CPU 12 relates to a relatively small area where the undesired presence is located, the processing is nearly two-dimensional. As such, in an alternative embodiment, the system technician may define a two-dimensional area instead of a three-dimensional volume where the undesired presence is located. In yet another embodiment, no volume or area is defined at all and CPU 12 scans all areas and volumes available through movement of joints 30₁, 30₂, 30₃ and 40. In still yet another embodiment, a three-dimensional image (not shown) of a generic body part may be defined in CPU 12. For example, CPU 12 will be given information relating to an area or volume of a predefined shape (e.g. an arm or leg). CPU 12 then uses this image to produce a predefined area or volume to be scanned.

At this point, at step S10, system 10 is actuated. At step S12, CPU 12 controls robotic arm control unit 26 to move robotic arm 28, and consequently laser and camera unit 20, so that camera 24 scans volume 38. Camera 24 scans volume 38 through a line of sight 42 between camera 24 and volume 38. The scanning can be effectuated by dividing volume 38 into a plurality of portions of a defined size. For example, if undesired presence 34 is hair with a diameter of 0.1 mm, and camera 24 produces a typical image of 500 by 500 pixels, the minimum sized scanning area will be typically 50 by 50 mm. Each portion of volume 38 is then sequentially analyzed by CPU 12.

Camera 24 forwards visual information that it processes concerning the image of each portion of volume 38 to CPU 12. CPU 12 uses image processing software to determine specific points of treatment in volume 38 where removal unit 22 can be applied to treat undesired presence 34. These points are stored in memory 46. Such software can be unique depending upon the nature of presence 34 (e.g. different algorithms for hair, pimples, moles, etc.) or can be a single algorithm designed to produce the most useful points of treatment in volume 38. For example, if undesired presence 34 is hair, CPU 12 will also determine an appropriate angle of incidence (not explicitly shown) which is most effective in removing hair. Such an angle of incidence will target the root or follicle of each hair based upon growth of the hair beneath the individual's skin. This growth can be determined by a portion of the hair which is external to the individual's skin. If body part 32 is shaved first, a portion of the hair extending externally from the individual will generally be oriented in the same direction as a portion of the hair beneath the individual's skin. The portion beneath the skin terminates in the root of the hair. CPU 12 extrapolates from this growth and determines a location of a hair root. Alternatively, ultrasound may be used to determine the orientation of hair beneath the individual's skin and consequently the location of the root.

Techniques such as contour finding technology may also be used to determine the location of an undesired presence. In contour finding technology, a processor reviews an image received by a camera and analyzes colors, contrasts between pixels, and shadows and determines boundaries between objects. The processor may then use deterministic programming or artificial intelligence learning to determine the location of an undesired presence. In deterministic programming, the processor is informed as to how the undesired presence will appear. In artificial intelligence learning, the processor is “taught” what the undesired presence looks like. If the undesired presence is, for example, a hair, by informing the processor of the color of the skin of body part 32 and the color of the hair, the processor can simply determine, based on colors, shapes, contours, and shadows, the location of the hair.

Each point of treatment in volume 38 is associated with a treatment position that removal unit 22 should assume with respect to undesired presence 34 so that when removal unit 22 is actuated to produce a laser beam 44, undesired presence 34 may be treated. Each treatment position is defined by the position of joints 30₁, 30₂, 30₃ and 40 and is determined by CPU 12 based upon the relative position of joints 30₁, 30₂, 30₃, 40, when camera 24 detects a corresponding point of treatment. CPU 12 also compensates for a distance d between camera 24 and laser 22 and incidence angle θ between line of sight 42 and laser beam 44—when determining each treatment position. The relative position of joints 30₁, 30₂, 30₃, 40 can be determined using, for example, rotary encoders, or any other position detection means. The treatment positions are also stored in memory 46.

The system technician can monitor the progress of CPU 12 and make modifications to calibrate system 10 for the specific body part 32. For example, the width of laser beam 44 can be adjusted in dependence upon the undesired presence 34.

At step S14, CPU 12 determines if robotic arm control unit 26 has moved camera 24 to scan all of volume 38. If camera 24 has not scanned all of volume 38, CPU 12 branches back to step S 12 and continues scanning. If camera 24 has scanned all of volume 38, CPU 12 branches to step S16.

At step S16, CPU 12 controls robotic arm control unit 26 and laser and camera unit 20 to move laser 22 to each treatment position determined above one at a time. An optional feedback control system may be used to ensure that laser and camera unit 20 is at the correct location. At step S17, CPU 12 may calibrate removal device 22 based on the characteristics of the undesired presence. For example, if the undesired presence is hair, a laser may be calibrated and used to remove the hair. Treatment or removal of an undesired presence is frequently more effective if one controls the duration of the laser's actuation or the wavelength of the laser based on characteristics of the undesired presence. For example, the color of a hair in contrast to the color of the body part (e.g. blonde hair or thicker hair may require longer exposure from a laser), or the color of a tatoo may be factored in calibrating removal device 22. As CPU 12 has already determined the location of undesired presences within volume 38, CPU 12 may also determine the most effective use of removal device 22 (i.e. duration, wavelength, etc.) based on the characteristics of the undesired presence. For example, once CPU 12 determines the characteristics of the undesired presence, CPU 12 may consult a lookup table in memory 46 to determine the most effective parameters for treatment device 22 to treat or remove undesired presence 34.

At step S18, CPU 12 then actuates laser 22 to treat the portion of undesired presence 34 at the respective point of treatment. At step S20, CPU 12 determines whether additional treatment positions are stored in memory 46. If additional treatment positions are stored in memory 46, CPU 12 branches back to step S16. If not, the operation of system 10 terminates.

As an example illustrating the operation of system 10, if undesired presence 34 is hair on an individual's arm, the technician will indicate a general volume 38 (or area) relating to a portion of arm 32 facing toward laser 40 and camera 24. CPU 12 then controls LCU 20 to scan volume 38. While CPU 12 scans volume 38, CPU 12 will determine the boundaries of arm 32 (e.g. if the arm is resting on a table, CPU 12 determines where arm 32 ends and the table begins). Laser 40 is then sequentially moved to the appropriate points of treatment and actuated. Arm 32 is then rotated or moved to expose a new volume 38 having undesired presence 34 and the process is repeated. In this way, complete treatment of an undesired presence on an individual is completed by performing treatment on one or more strips or longitudinal sections of body part 32.

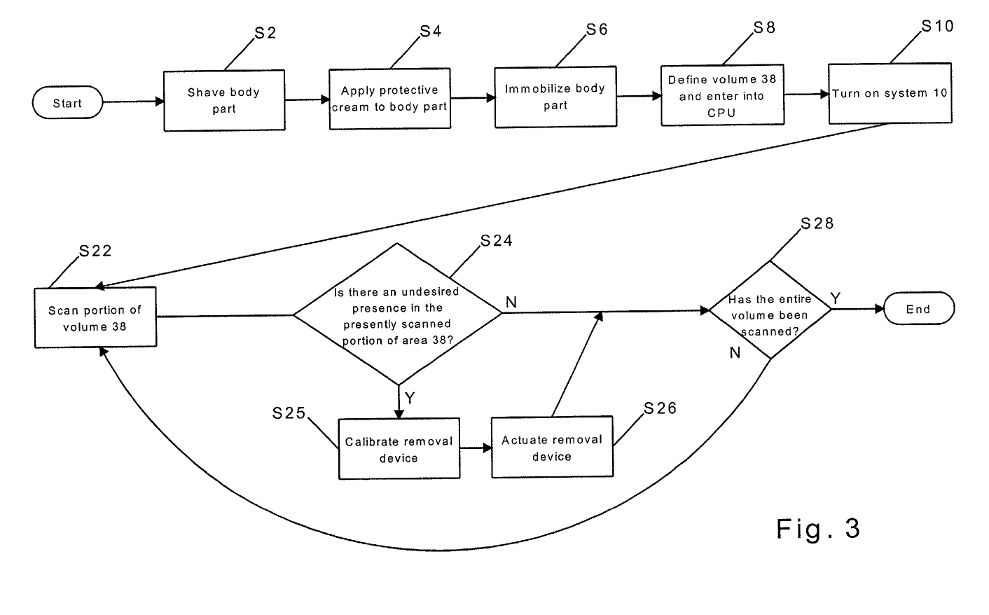

A second embodiment of the invention is shown in FIG. 3. In the second embodiment, camera 24 is aligned and focused with respect to laser and camera unit 20 so that line of sight 42 and laser 44 reach the same point on body part 32. This is beneficial in that CPU 12 can account for some movement of body part 32 and provide real time information to laser and camera unit 20. In this second embodiment, steps S2-S10 are substantially the same in function as with the first embodiment and so description of these steps is omitted for brevity.

After system 10 is turned on in step S10, at step S22, CPU 12 controls robotic arm control unit 26 to move camera 24 to scan a portion of volume 38. The data from camera 24 is immediately processed by CPU 12 and at step S24, CPU 12 determines whether an undesired presence is located within the currently scanned portion of volume 38. If an undesired presence is within the currently scanned portion of volume 38, CPU 12 branches control to step S25, where CPU 12 optionally calibrates removal device 22 as discussed above with reference to step S17 in the previous embodiment. Thereafter, CPU 12 branches to step S26 and actuates laser 22. If necessary, CPU 12 may move robotic arm control unit 26 or one of joints 30₁, 30₂, 30₃, or 40 to produce an optimally placed laser beam 22. For example, as discussed above in the previous embodiment, an angle of incidence may be determined based on growth of hair and the location of a hair root. Moreover, if multiple undesired presences are within the current scanned portion of volume 38, laser beam 22 may need to be moved and actuated multiple times. After actuation of laser 22 in step S26, CPU 12 branches control to step S28. If there is no undesired presence within the currently scanned portion of volume 38, at step S24, CPU 12 also branches control to step S28.

At step S28, CPU 12 determines whether all of volume 38 has been scanned. If portions of volume 38 have not been scanned, control branches back to step S22 and a new portion of volume 38 is scanned. If all of volume 38 has been scanned, the operation of system 10 terminates. In this way, CPU 12 has real-time information of where laser 40 is incident upon body part 32. This helps ensure accurate application of laser 40 upon undesired presence 34. It also enables CPU 12 to place camera 24 at a desired focal length with respect to undesired presence 34.

Although a single laser and camera are shown, clearly additional cameras and lasers could be added to increase the operational speed of the device. Other technology, besides a laser, could be used to treat the undesired presence. For example, tweezers, electrotweezers, a device which can electrocute the undesired presence, a device which pours a chemical substance (effective to treat the undesired presence) on the undesired presence, or any other device which can cause treatment of an undesired presence is within the scope of the invention. The term camera is intended to refer to any imaging unit.

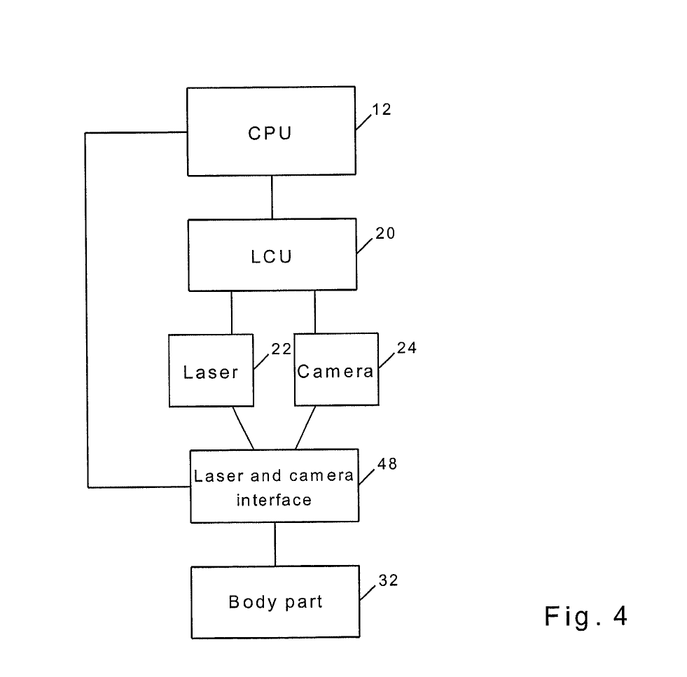

Although laser and camera unit 20 is shown coupled to a robotic arm, clearly any structure which allows CPU 12 to control the imaging of camera 24 and the location where laser 22 impinges upon body part 32 is within the scope of the invention. For example, as is shown in FIG. 4, laser 22 and camera 24 can remain stationary and CPU 12 may optionally control a laser and camera interface 48 to alter the location where camera 24 scans and where laser 22 impinges. Interface 48 could be used for either one or both of laser 22 and camera 24. Interface 48 could include, for example, mirrors that control the termination of line of sight 42 and/or laser beam 44.

Thus, by providing data relating to treatment locations, control of a robotic arm to move a laser or other treatment device to provide treatment at the treatment locations, and control of the actuation of the laser, to a central processing unit, a faster, less expensive, more efficient and more precise undesired presence treatment system is possible than that available in the prior art.

While preferred embodiments of the invention have been disclosed, various modes of carrying out the principles disclosed herein are contemplated as being within the scope of the following claims. Therefore, it is understood that the scope of the invention is not to be limited except as otherwise set forth in the claims.

What is claimed is:

1. A method for treating an undesired presence in or on an individual using a central processing unit, said method comprising:
defining at least a scanning area on said individual, said scanning area including said undesired presence;
scanning a portion of said scanning area with an imaging unit controlled by said central processing unit;
determining, based on said scanning, whether said portion includes said undesired presence;
controlling, by said central processing unit, a treatment unit to treat said undesired presence, when said portion includes said undesired presence; and
repeating said steps of scanning, determining, and controlling until all portions of said scanning area are scanned.

2. The method as recited in claim 1, further comprising the step of calibrating the treatment unit based on a characteristic of said undesired presence, before said step of controlling.

3. The method as recited in claim 1, wherein said treatment unit is at least one of a laser, tweezers, electrotweezers, and a chemical effective to treat said undesired presence.

4. The method as recited in claim 1, wherein said undesired presence is hair.

5. The method as recited in claim 4, further comprising the step of dying said hair before performing said step of scanning.

6. The method as recited in claim 1, wherein said undesired presence is at least one of a mole, tatoo, pimple, scar tissue, wart, and a freckle.

7. The method as recited in claim 1, further comprising the step of shaving said individual before said step of defining.

8. The method as recited in claim 1, further comprising the step of immobilizing said individual before said step of defining.

9. The method as recited in claim 1, wherein said area is a three-dimensional volume.

10. The method as recited in claim 1, wherein said steps of scanning and controlling are performed through use of a robotic arm.

11. The method as recited in claim 10, wherein said area is the locus of all locations available through movement of said robotic arm.

12. The method as recited in claim 1, wherein said steps of scanning and controlling are performed through control of an interface coupled to said imaging unit and said treatment unit.

13. The method as recited in claim 1, wherein said area is defined by an image of a generic body part.

14. The method as recited in claim 1, further comprising the steps of:
moving a body part on said individual including said undesired presence; and thereafter, performing said steps of defining, scanning, determining, controlling, and repeating.

15. A method for treating an undesired presence in or on an individual using a central processing unit, said method comprising:
defining at least a scanning area on said individual, said scanning area including said undesired presence;
scanning a portion of said scanning area with an imaging unit controlled by said central processing unit;
determining, based on said scanning, whether said portion includes said undesired presence, when said portion includes said undesired presence;
calculating a point of treatment where said treatment unit is effective to treat said undesired presence when said portion includes said undesired presence;
first repeating said steps of scanning, determining, and calculating until all portions of said scanning volume are scanned;
controlling said treatment unit, by said central processing unit, to act upon one of said points of treatment; and
second repeating said step of controlling for all of said points of treatment.

16. The method as recited in claim 15, further comprising the step of calibrating the treatment unit based on a characteristic of said undesired presence, before said step of controlling.

17. The method as recited in claim 15, wherein said treatment unit is at least one of a laser, tweezers, electrotweezers, and a chemical effective to treat said undesired presence.

18. The method as recited in claim 15, wherein said undesired presence is hair.

19. The method as recited in claim 18, further comprising the step of dying said hair before performing said step of scanning.

20. The method as recited in claim 15, wherein said undesired presence is at least one of a mole, tatoo, pimple, scar tissue, wart, and a freckle.

21. The method as recited in claim 15, further comprising the step of shaving said individual before said step of defining.

22. The method as recited in claim 15, further comprising the step of immobilizing said individual before said step of defining.

23. The method as recited in claim 15, wherein said area is a three-dimensional volume.

24. The method as recited in claim 15, wherein said steps of scanning and controlling are performed through use of a robotic arm.

25. The method as recited in claim 24, wherein said area is the locus of all locations available through movement of said robotic arm.

26. The method as recited in claim 15, wherein said steps of scanning and controlling are performed through control of an interface coupled to said imaging unit and said treatment unit.

27. The method as recited in claim 15, wherein said area is defined by an image of a generic body part.

28. The method as recited in claim 15, further comprising the steps of:
moving a body part on said individual including said undesired presence; and thereafter,
performing said steps of defining, scanning, determining, calculating, first repeating controlling, and second repeating.

29. An undesired presence treatment system for treating an undesired presence in or on an individual, said system comprising:
a central processing unit;
an imaging unit coupled to said central processing unit; and
a treatment unit coupled to said central processing unit; wherein
said central processing unit includes data for performing the steps of:
defining at least a scanning area on said individual, said scanning area including said undesired presence;
scanning a portion of said scanning area with an imaging unit;
determining, based on said scanning, whether said portion includes said undesired presence;
controlling said treatment unit to treat said undesired presence, when said portion includes said undesired presence; and
repeating said steps of scanning, determining, and controlling until all portions of said scanning volume are scanned.

30. The system as recited in claim 29, wherein said central processing unit further includes data for performing the step of calibrating the treatment unit based on a characteristic of said undesired presence, before said step of controlling.

31. The system as recited in claim 29, wherein said treatment unit is at least one of a laser, tweezers, electrotweezers, and a chemical effective to treat said undesired presence.

32. The system as recited in claim 29, wherein said undesired presence is hair.

33. The system as recited in claim 29, wherein said undesired presence is at least one of a mole, tatoo, pimple, scar tissue, wart, and a freckle.

34. The system as recited in claim 29, wherein said area is a three-dimensional volume.

35. The system as recited in claim 29, further comprising a robotic arm coupled to said imaging unit, treatment unit, and central processing unit; and wherein
said steps of scanning and controlling are performed through the use of said robotic arm.

36. The system as recited in claim 35, wherein said area is the locus of all locations available through movement of said robotic arm.

37. The system as claimed in claim 29, further comprising an interface coupled to said imaging unit, treatment unit, and central processing unit; and wherein
said steps of scanning and controlling are performed through the use of said interface.

38. The system as recited in claim 29, wherein said area is defined by an image of a generic body part.

39. The system as recited in claim 29, wherein:
said treatment unit includes a plurality of lasers; and
said imaging unit includes a plurality of cameras.

40. The system as recited in claim 29, wherein said imaging unit and said treatment unit are coupled to said central processing unit through a wireless communication.

41. An undesired presence treatment system for treating an undesired presence in or on an individual, said system comprising:
a central processing unit;
an imaging unit coupled to said central processing unit; and
a treatment unit coupled to said central processing unit; wherein
said central processing unit contains data for performing the steps of:
defining at least a scanning area on said individual, said scanning area including said undesired presence;
scanning a portion of said scanning area with said imaging unit;
determining, based on said scanning, whether said portion includes said undesired presence;
calculating a point of treatment where said treatment unit is effective to treat said undesired presence, when said portion includes said undesired presence;
first repeating said steps of scanning, determining and calculating until all portions of said scanning area are scanned;
controlling said treatment unit to act upon one of said points of treatment; and
second repeating said step of controlling for all of said points of treatment.

42. The system as recited in claim 41, wherein said central processing unit further includes data for performing the step of calibrating the treatment unit based on a characteristic of said undesired presence, before said step of controlling.

43. The system as recited in claim 41, wherein said treatment unit is at least one of a laser, tweezers, electrotweezers, and a chemical effective to treat said undesired presence.

44. The system as recited in claim 41, wherein said undesired presence is hair.

45. The system as recited in claim 41, wherein said undesired presence is at least one of a mole, tatoo, pimple, scar tissue, wart, and a freckle.

46. The system as recited in claim 45, wherein said area is a three-dimensional volume.

47. The system as recited in claim 45, further comprising a robotic arm coupled to said imaging unit, treatment unit, and central processing unit; and wherein
said steps of scanning and controlling are performed through the use of said robotic arm.

48. The system as recited in claim 47, wherein said area is the locus of all locations available through movement of said robotic arm.

49. The system as claimed in claim 45, further comprising an interface coupled to said imaging unit, treatment unit, and central processing unit; and wherein
said steps of scanning and controlling are performed through the use of said interface.

50. The system as recited in claim 45, wherein said area is defined by an image of a generic body part.

51. The system as recited in claim 45, wherein:
said treatment unit includes a plurality of lasers; and
said imaging unit includes a plurality of cameras.

52. The system as recited in claim 45, wherein said imaging unit and said treatment unit are coupled to said central processing unit through a wireless communication.
